(19) United States
(12) Patent Application Publication
(10) Pub. No.: US 2025/0032111 A1
(43) Pub. Date: Jan. 30, 2025

(54) EXPANDABLE TISSUE ENGAGEMENT APPARATUS AND METHOD

(71) Applicant: Teleflex Life Sciences LLC, Wilmington, DE (US)

(72) Inventors: Theodore C. Lamson, Pleasanton, CA (US); Theodore Bender, San Anselmo, CA (US); Jolene Cutts, San Francisco, CA (US); Robert M. George, San Jose, CA (US); Jennifer Kiyoi, Pleasanton, CA (US); Daniel Merrick, Dublin, CA (US); Ailee Pham, Milpitas, CA (US); Maheshwara Rao, Oakland, CA (US); Christopher Zaler, Los Gatos, CA (US); Curtis Yarra, Oakland, CA (US); Kevin Alexander Lessard, San Francisco, CA (US)

(73) Assignee: Teleflex Life Sciences LLC, Wilmington, DE (US)

(21) Appl. No.: 18/914,997

(22) Filed: Oct. 14, 2024

Related U.S. Application Data
(63) Continuation of application No. 18/188,942, filed on Mar. 23, 2023, now Pat. No. 12,121,228.
Application No. 18/188,942 is a continuation of application No. 16/577,013, filed on Sep. 20, 2019, now Pat. No. 11,672,520.
Application No. 16/577,013 is a continuation of application No. PCT/US2018/067229, filed on Dec. 21, 2018.
(60) Provisional application No. 62/610,184, filed on Dec. 23, 2017.

(51) Int. Cl. A61B 17/02; A61B 17/00; A61B 17/04; A61B 17/22; A61B 17/34; A61B 90/00; A61M 29/00

(52) U.S. Cl. CPC A61B 17/0218; A61B 17/3478; A61M 29/00; A61B 2017/00274; A61B 2017/00858; A61B 2017/00951; A61B 2017/0409; A61B 2017/22072; A61B 2090/0811; A61M 2210/166

(57) ABSTRACT

A system and associated method for manipulating tissues and anatomical or other structures in medical applications for the purpose of treating diseases or disorders or other purposes. In one aspect, the system includes an expandable structure for enhancing engagement with median lobe prostate tissue.

### CROSS REFERENCE TO RELATED APPLICATIONS

The present application is a continuation of U.S. application Ser. No. 18/188,942,filed Mar. 23, 2023, which is a continuation of U.S. application Ser. No. 16/577,013 filed Sep. 20, 2019, now U.S. Pat. No. 11,672,520, which is a continuation of PCT Application Serial No. PCT/US2018/067229 filed Dec. 21, 2018, which claims priority to U.S. Provisional Application Ser. No. 62/610,184, filed Dec. 23, 2017, entitled “Median Lobe Engagement Apparatus and Method,” each of which is incorporated herein by reference.

### FIELD OF THE DISCLOSURE

The present disclosure relates generally to medical devices and methods, and more particularly to systems and associated methods for manipulating or engaging tissues and anatomical or other structures within the body of human or animal subjects for the purpose of treating diseases or disorders.

One example of a condition where it is desirable to lift, compress, or otherwise remove a pathologically enlarged tissue is Benign Prostatic Hyperplasia (BPH). BPH is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout a man's life. In some men, the prostatic capsule around the prostate gland may prevent the prostate gland from enlarging further. This causes the inner region of the prostate gland to squeeze the urethra as a result of the gland enlarging. This pressure on the urethra increases resistance to urine flow through the region of the urethra enclosed by the prostate. Thus, the urinary bladder has to exert more pressure to force urine through the increased resistance of the urethra. Chronic over-exertion causes the muscular walls of the urinary bladder to remodel and become stiffer. This combination of increased urethral resistance to urine flow and hypertrophy and stiffness of urinary bladder walls leads to a variety of lower urinary tract symptoms (LUTS) that may severely reduce the patient's quality of life. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, and urgent need to urinate.

In addition to being present in patients with BPH, LUTS may also be present in patients with prostate cancer, prostate infections, and chronic use of certain medications (e.g. ephedrine, pseudoephedrine, phenylpropanolamine, and antihistamines such as diphenhydramine or chlorpheniramine) that cause urinary retention especially in men with prostate enlargement.

Although BPH is rarely life threatening, it can lead to numerous clinical conditions including urinary retention, renal insufficiency, recurrent urinary tract infection, incontinence, hematuria, and bladder stones.

In developed countries, a large percentage of the patient population undergoes treatment for BPH symptoms. It has been estimated that by the age of 80 years, approximately 25% of the male population of the United States will have undergone some form of BPH treatment. At present, the available treatment options for BPH include watchful waiting, medications (phytotherapy and prescription medications), surgery, and minimally invasive procedures.

For patients who choose the watchful waiting option, no immediate treatment is provided to the patient, but the patient undergoes regular exams to monitor progression of the disease. This is usually done on patients who have minimal symptoms that are not especially bothersome.

Medical procedures for treating BPH symptoms include Transurethal Resection of the Prostate (TURP), Transurethral Electrovaporization of the Prostate (TVP), Transurethral Incision of the Prostate (TUIP), Laser Prostatectomy, Open Prostatectomy, Transurethral Microwave Thermotherapy (TUMT), Transurethral Needle Ablation (TUNA), Interstitial Laser Coagulation (ILC), and Prostatic Stents.

The most effective current methods of treating BPH in terms of relieving the symptoms of BPH also carry a high risk of adverse effects. These methods may require general or spinal anesthesia and/or may have potential adverse effects that dictate that the procedures be performed in a surgical operating room, followed by a hospital stay for the patient. The methods of treating BPH that carry a lower risk of adverse effects are also associated with a lower reduction in the symptom score. While several of these procedures can be conducted with local analgesia in an office setting, the patient does not experience immediate relief and, in fact, often experiences worse symptoms for weeks after the procedure until the body begins to heal. Additionally, many surgical or minimally invasive approaches require a urethral catheter to be placed in the bladder, and in some cases left in the bladder for weeks. In some cases, catheterization is indicated because the therapy actually causes obstruction during a period of time post operatively, and in other cases it is indicated because of post-operative bleeding and the potential for the formation of occlusive clots. While drug therapies are easy to administer, the results are frequently suboptimal, take significant time to take effect, and often include undesirable side effects.

There have been advances in developing minimally invasive devices and methods for displacing and/or compress lobes of a prostate gland to receive pressure on and provide a less obstructed path through a urethra. These methods have focused on treating the lateral lobes of the prostate gland. There remains, however, a need for the development of new devices and methods that can be used for various procedures where it is desired to lift, compress, support, or reposition the median lobe of a prostate in a discrete procedure or in combination with treating BPH. In particular, there is a need for alternative apparatus and treatment approaches for the purpose of manipulating the median lobe of a prostate.

Still further, there is an ongoing need in the field of minimally invasive medical devices for devices and methods for the manipulation of tissue in other parts of the anatomy.

The present disclosure addresses these and other needs.

### SUMMARY

Embodiments of the invention include a treatment device for engaging and manipulating a median lobe of a prostate gland. The treatment device includes an elongate tissue access assembly coupled to a handle assembly, wherein the elongate tissue access assembly is configured to be inserted within an introducer sheath, and a tissue engagement structure attached to a distal end portion of the elongate tissue access assembly, wherein the tissue engagement structure can transition from a contracted state to an expanded state when the elongate tissue access assembly exits a distal end of the introducer sheath.

In another embodiment of the invention, the tissue engagement structure comprises a first expandable portion having an asymmetrical cross-section. In another embodiment of the invention, a proximal portion of the first expandable portion is attached to the elongate tissue access assembly. In another embodiment of the invention, the tissue engagement structure comprises a channel to receive the distal end portion of the elongate tissue access assembly. In another embodiment of the invention, movement of the tissue engagement structure relative to the elongate tissue access assembly is constrained to be along a longitudinal axis of the elongate tissue access assembly. In another embodiment of the invention, movement of the tissue engagement structure relative to the elongate tissue access assembly transitions the tissue engagement structure from the contracted state to the expanded state. In another embodiment of the invention, the elongate tissue access assembly further comprises an aperture and a needle assembly that is extendable through the aperture. In another embodiment of the invention, the tissue engagement structure further comprises a first visual marker indicating a tissue entry position for the needle assembly. In another embodiment of the invention, the tissue engagement structure further comprises a second expandable portion having an asymmetrical cross-section. In another embodiment of the invention, wherein the elongate tissue access assembly further comprises an aperture and a needle assembly that is extendable through the aperture.

Embodiments of the invention include, a system for engaging and manipulating a median lobe of a prostate gland such that the system includes an anchor delivery device comprising an elongate tissue access assembly, wherein the elongate tissue access assembly is configured to be inserted within an introducer sheath, a tissue anchor housed within the anchor delivery device, and a tissue engagement structure attached to a distal end portion of the elongate tissue access assembly, wherein the tissue engagement structure can transition from a contracted state to an expanded state. In another embodiment of the invention, the tissue engagement structure comprises a first expandable portion having an asymmetrical cross-section. In another embodiment of the invention, a proximal portion of the first expandable portion is fixedly attached to the elongate tissue access assembly. In another embodiment of the invention, the tissue engagement structure comprises a slidable portion coupled to the elongate tissue access assembly. In another embodiment of the invention, movement of the tissue engagement structure relative to the elongate tissue access assembly transitions the tissue engagement structure from the contracted state to the expanded state. In another embodiment of the invention, the anchor delivery device further comprises a needle assembly. In another embodiment of the invention, the needle assembly is configured to deliver the tissue anchor. In another embodiment of the invention, the tissue engagement structure comprises a first expandable portion. In another embodiment of the invention, the first expandable portion further comprises a first visual marker indicating a tissue entry position for the needle assembly. In another embodiment of the invention, the tissue engagement structure further comprises a second expandable portion.

Other features and advantages of the present disclosure will become apparent from the following detailed description, taken in conjunction with the accompanying drawings, which illustrate, by way of example, the principles of the disclosure.

### BRIEF DESCRIPTION OF THE DRAWINGS

FIG. 1 is a cross-sectional view, depicting anatomy in the area surrounding a prostate in a human subject.

FIG. 2 is an enlarged cross-sectional view of an area of FIG. 1, depicting anatomy immediately surrounding and adjacent a prostate.

FIG. 3 is a schematic view, depicting prostatic anatomy zones.

FIG. 4 is a schematic cross-sectional view, depicting further details of the anatomy zones shown in FIG. 3.

FIG. 5 is a cross-sectional view, depicting a normal prostate.

FIG. 6 is a cross-sectional view, depicting a prostate with enlarged lateral lobes.

FIG. 7 is a cross-sectional view, depicting a prostate with enlarged lateral lobes and an enlarged median lobe.

FIG. 8A is a side view, depicting an embodiment of a delivery device;

FIGS. 8B is an enlarged perspective view, depicting the distal end of an embodiment of a delivery device.

FIG. 9A is a top view, depicting an embodiment of an expandable structure that can be employed to engage and manipulate tissue.

FIG. 9B is a perspective view, depicting an embodiment of an expandable structure that can be employed to engage and manipulate tissue.

FIG. 9C is an enlarged view, depicting an embodiment of an expandable engagement and manipulation device mounted on the distal end of an embodiment of a delivery device.

FIG. 10 is a perspective view, depicting another embodiment of an expandable structure that can be employed to engage and manipulate tissue.

FIG. 11 is a perspective view, depicting another embodiment of an expandable structure that can be employed to engage and manipulate tissue.

FIG. 12A is a top perspective view, depicting an embodiment of an expandable engagement and manipulation device.

FIG. 12B is an enlarged top perspective view, depicting a distal end portion of an expandable engagement and manipulation device.

FIG. 12C is a perspective view, depicting another embodiment of an expandable structure that can be employed to engage and manipulate tissue.

FIG. 12D is an enlarged bottom perspective view, depicting a distal end portion of an embodiment of an expandable engagement and manipulation device.

FIG. 12E is an enlarged bottom view, depicting a distal end portion of an embodiment of an expandable engagement and manipulation device.

FIG. 12F is an enlarged side view, depicting a distal end portion of an embodiment of an expandable engagement and manipulation device.

FIG. 12G is a cross-sectional view, depicting a distal end portion of an embodiment of an expandable engagement and manipulation device.

FIG. 13A is a top view, depicting an embodiment of an expandable structure with telescoping arms that can be employed to engage and manipulate tissue.

FIGS. 13B-D are various partial perspective views, depicting telescoping arms of an expandable structure that can be employed to engage and manipulate tissuc.

FIGS. 14A-B are cross-sectional views, depicting alternative configurations for telescoping arms.

FIGS. 15A-D are various cross-sectional views, depicting details of an approach to engaging, compressing and manipulating a median prostate lobe of a prostate.

FIG. 16A is a perspective view, depicting a distal end portion of an expandable structure that can be employed to engage and manipulate tissue housed in a sheath.

FIG. 16B is a perspective view, depicting the distal end portion of the expandable structure of FIG. 16A in an expanded state after exiting the sheath.

FIG. 17A is a perspective view, depicting the distal end of a sheath.

FIG. 17B is a perspective view, depicting an expanded structure that can be employed to engage and manipulate tissue attached to a sheath.

FIG. 18A is a perspective view, depicting an embodiment of an expandable wire structure that can be employed to engage and manipulate tissue, in a contracted state.

FIG. 18B is a perspective view, depicting an embodiment of an expandable wire structure that can be employed to engage and manipulate tissue, in an expanded state.

FIG. 19A is a perspective view, depicting another embodiment of an expandable wire structure that can be employed to engage and manipulate tissue, in a contracted state.

FIGS. 19B is a perspective view, depicting another embodiment of an expandable wire structure that can be employed to engage and manipulate tissue, in an expanded state.

FIGS. 20A-C are enlarged perspective views, depicting a distal end portion of an expandable engagement and manipulation device incorporating various supplemental tissue engagement structures.

FIGS. 20D-F are enlarged perspective views, depicting a distal end portion of a tissue access and a device incorporating various supplemental tissue engagement structures.

FIGS. 20G-H are enlarged views, depicting structures that can be incorporated into the distal end portion of a tissue engagement and manipulation device.

FIG. 21 is a partial cross-sectional view, depicting yet another approach to engaging and manipulating target tissue.

FIG. 22 is an enlarged view, depicting further structure incorporated into an engagement and manipulation device.

FIG. 23 is an enlarged view, depicting supplemental structure forming part of an engagement and manipulation device.

FIGS. 24A-B are enlarged views, depicting structures that can be incorporated into the distal end portion of a tissue engagement and manipulation device and methods for engaging and manipulating tissuc.

FIGS. 25A-B are enlarged views, depicting structures that can be incorporated into the distal end portion of a tissue engagement and manipulation device.

FIGS. 26A-B are enlarged views, depicting structures that can be incorporated into the handle portion of a tissue engagement and manipulation device.

FIGS. 27A-B are enlarged views, depicting other structures that can be incorporated into the distal end portion of a tissue engagement and manipulation device.

### DETAILED DESCRIPTION OF THE SEVERAL DRAWINGS

Turning now to the figures, which are provided by way of example and not limitation, the present disclosure is directed to a device configured to engage and manipulate tissue within a patient's body for treatment purposes. The disclosed apparatus can be employed for various medical purposes including but not limited to retracting, lifting, compressing, approximating, supporting, and/or repositioning tissues, organs, anatomical structures, grafts, or other material found within a patient's body. Such tissue manipulation is intended to facilitate the treatment of diseases or disorders, including, but not limited to, the displacement, compression and/or retraction of the median lobe of a prostate.

In an aspect of the present disclosure, the tissue engagement or manipulation forms the primary interventional procedure. In other aspects, the tissue engagement or manipulation forms one portion of an interventional procedure, such as the treatment of BPH or for the purpose of retracting, lifting, compressing, approximating, supporting or repositioning other anatomy or for the purpose of retracting, lifting, compressing, approximating, supporting, or repositioning a first section of anatomy with respect to a second section of anatomy.

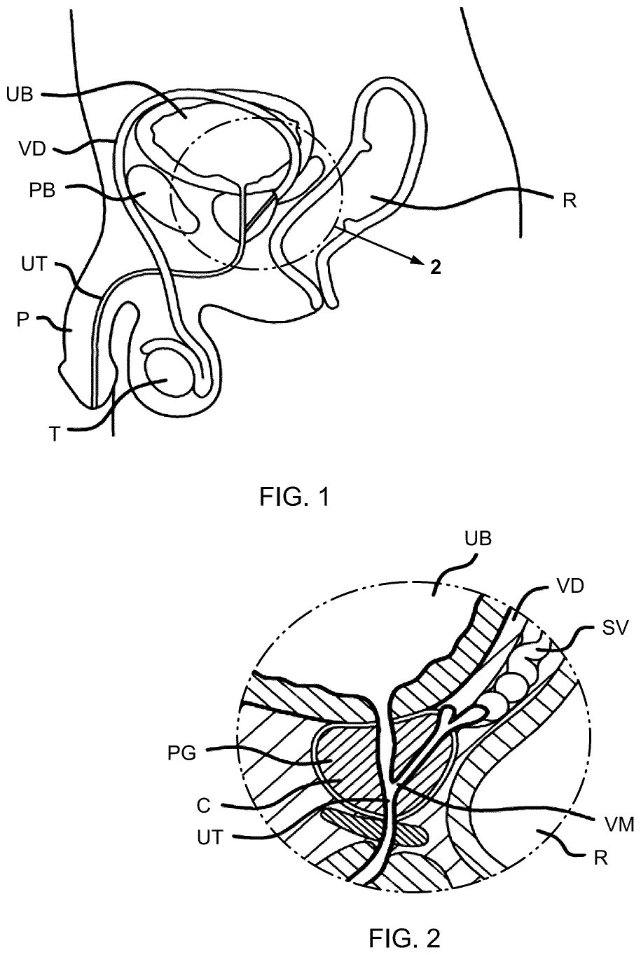

With reference to FIGS. 1-4, various features of urological anatomy of a human male subject are presented. The prostate gland PG is a walnut-sized gland located adjacent the urinary bladder UB. The urethra UT runs through the prostate gland PG and the penis P. The prostate gland PG secretes fluid that protects and nourishes sperm. The prostate gland PG also contracts during ejaculation to expel semen and to provide a valve to keep urine out of the semen. A capsule C surrounds the prostate gland PG.

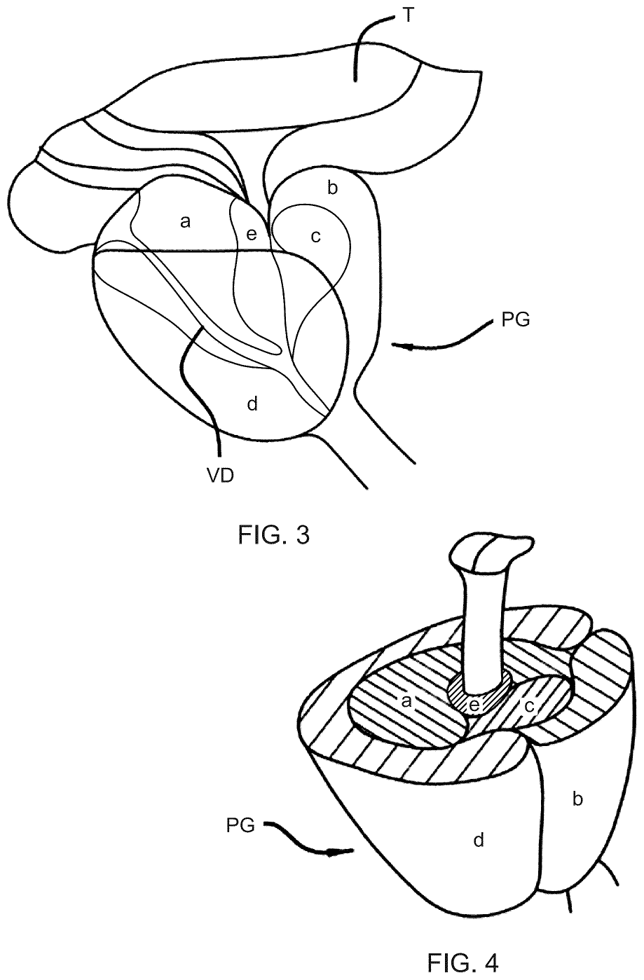

The urinary bladder UB holds urine. The vas deferentia VD define ducts through which semen is carried, and the seminal vesicles SV secrete seminal fluid. The rectum R is the end segment of the large intestine through which waste is dispelled. The urethra UT carries both urine and semen out of the body. Thus, the urethra is connected to the urinary bladder UB and provides a passageway to the vas deferentia VD and seminal vesicles SV. The verumontanum VM is a crest in the wall of the urethra UT where the seminal ducts enter. The prostatic urethra is the section of the urethra UT which extends through the prostate. The trigone T (see FIG. 3) is a smooth triangular region of the bladder. It is sensitive to expansion and signals the brain when the urinary bladder UB is full.

The prostate gland can be classified by zones or described by referring to its lobes (See FIG. 4). Whereas the zone classification is typically used in pathology, the lobe classification is more often used in anatomy. The central zone (a) of a prostate gland PG is about 25% of a normal prostate and this zone surrounds the ejaculating ducts. There is some prevalence of benign prostate hyperplasia in the transition zone. The fibromuscular zone (b) is usually devoid of glandular components and, as its name suggests, is composed of muscle tissue and fibrous tissue. The transitional zone (c) generally overlays the proximal urethra and is the region of the gland that grows throughout life. This zone is often associated with the condition of benign prostatic enlargement. Finally, the peripheral zone (d) is the sub-capsular portion of the posterior aspect of the prostate gland that surrounds the distal urethra.

The lobe characterization is different from the zone characterization, but there is some overlap. The anterior lobe is devoid of glandular tissue and is formed of fibromuscular tissue. The anterior lobe roughly corresponds to the anterior portion of the transitional zone (c). The posterior lobe roughly corresponds to the peripheral zone (d) and can be palpated through the rectum during a digital rectal exam. The posterior lobe is the site of 70-80% of prostatic cancers. The lateral lobes are the main mass of the prostate and are separated by the urethra. All pathological zones may be present in the lateral lobes. Lastly, the median lobe roughly corresponds to part of the central zone. It varies greatly in size from subject to subject and in some cases is devoid of glandular tissue.

A large or enlarged median lobe can act as a ball valve, blocking the bladder neck, or opening, into the urethra. Various approaches are contemplated to address such a condition. It is contemplated that the median lobe can be compressed, displaced and/or retracted to eliminate or decrease the blocking of the bladder neck opening.

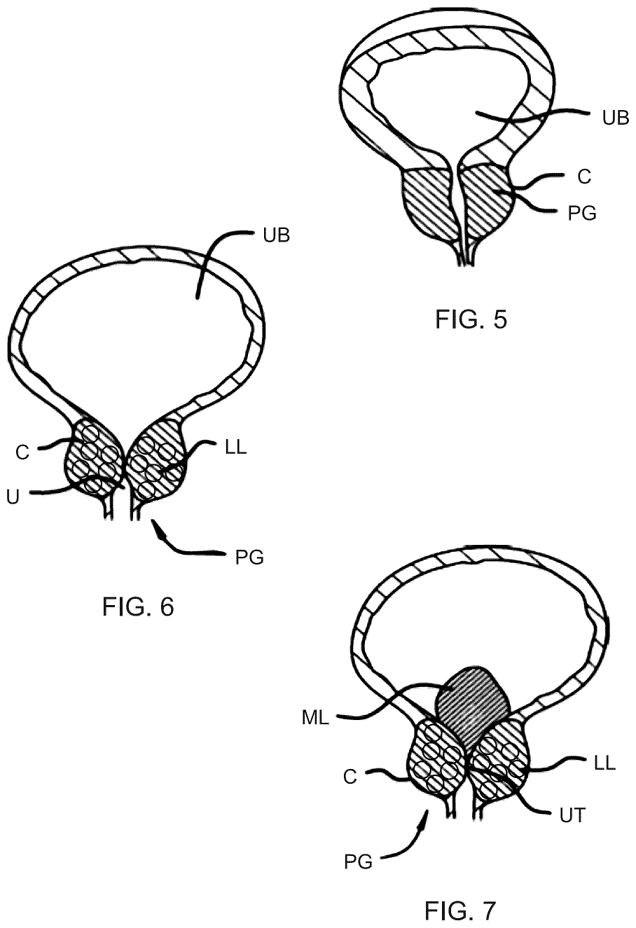

Turning now to FIGS. 5-7, there are shown various prostate glands in cross-section. FIG. 5 depicts the urinary bladder UB and prostate gland PG of a healthy subject. FIG. 6 illustrates an individual with a prostate having enlarged lateral lobes LL and FIG. 7 depicts a subject suffering from both enlarged lateral lobes LL and an enlarged median lobe ML. It is to be appreciated that such enlarged anatomy impinges on the urethra UT and affects normal bladder functioning. The following devices and approaches can be employed to access and manipulate prostatic tissue during an interventional or diagnostic procedure.

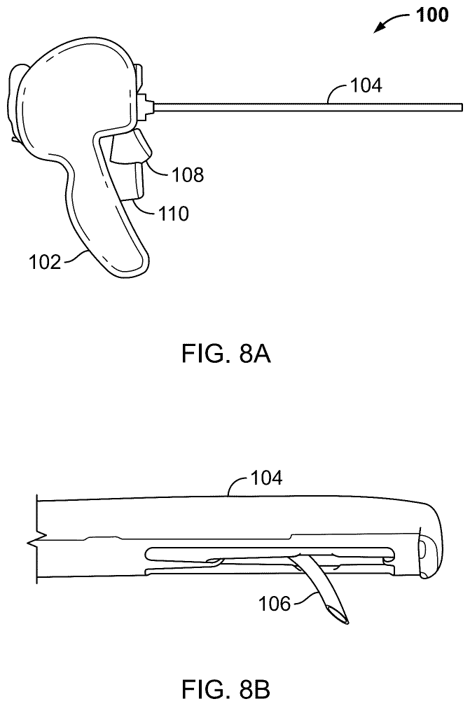

Referring now to FIGS. 8A & 8B, an embodiment of a treatment device 100 is shown. Treatment device 100 can include a handle assembly 102 and an elongate tissue access assembly 104. Elongate assembly 104 can be configured to access an interventional site as well as engage and manipulate target tissue. Treatment device 100 can be configured to assemble and implant one or more anchor assemblies or implants within a patient's body. The device is further contemplated to be compatible for use with minimally invasive techniques (such as cystoscopy) such that a patient can tolerate a procedure while awake or under light sedation rather than under general anesthesia. The device additionally includes structures configured to receive a conventional remote viewing device (c.g., an endoscope) so that the steps being performed at the interventional site can be observed by the physician.

The elongate assembly 104 can house members to manipulate target tissue including, but not limited to, a needle assembly 106. Elongate assembly 104 can also be equipped with features to manipulate target tissue and/or stabilize the device at its interventional site. Elongate assembly 104 can be inserted through a sheath of a size compatible with conventional cystoscopy, including sizes such as 19F or 21F. Elongate assembly 104 can be rigid or flexible. In some preferred embodiments, elongate assembly 104 is sufficiently rigid (or can be made sufficiently rigid when at the interventional site) to allow manual compression of tissue at an interventional site by leveraging or pushing handle assembly 102. Various embodiments of treatment device 100 can include subassemblies and components to dissect, resect, or otherwise alter a prostatic lobe.

In one particular, non-limiting use in treating a prostate, the elongate tissue access assembly of a delivery device is placed within a urethra leading to a urinary bladder of a patient. In one approach, the delivery device can be placed within an introducer sheath previously positioned in the urethra or alternatively, the delivery device can be inserted directly within the urethra. When employing an introducer sheath, the sheath can be attached to a sheath mount assembly. The sheath is advanced within the patient until a leading end thereof reaches a prostate gland. In a specific approach, a first side (i.e., lateral lobe) of the prostate to be treated is chosen while the device extends through the bladder and the device is turned accordingly. The distal end of the elongate tissue access assembly can be used to depress the urethra into the prostate gland by compressing the inner prostate tissue. The inside of the prostate gland (i.e., adenoma) is spongy and compressible and the outer surface (i.e., capsule) of the prostate gland is firm. By pivoting the elongate tissue access assembly laterally about the pubic symphysis PS relative to the patient's midline., the physician can depress the urethra into the prostate gland compressing the adenoma and creating the desired opening through the urethra. Further details and background concerning related and complementary interventional procedures are described in various U.S. patents, including U.S. Pat. Nos. 8,491,606 and 8,758,366, the entirety of the contents of which are hereby incorporated by reference.

When the treatment device is used at an interventional site, such as the median lobe of the prostate, prior to deployment of an implant or alteration of prostatic tissue the median lobe often requires manipulation into a position conducive to receiving an implant. Embodiments of a device and method of use that can position and stabilize a treatment device to better engage and manipulate target tissue, including the median lobe of the prostate, are described below. In a preferred embodiment, such a device can include a winged, expandable/collapsible structure that increases the distal surface area of the device to engage and manipulate tissue.

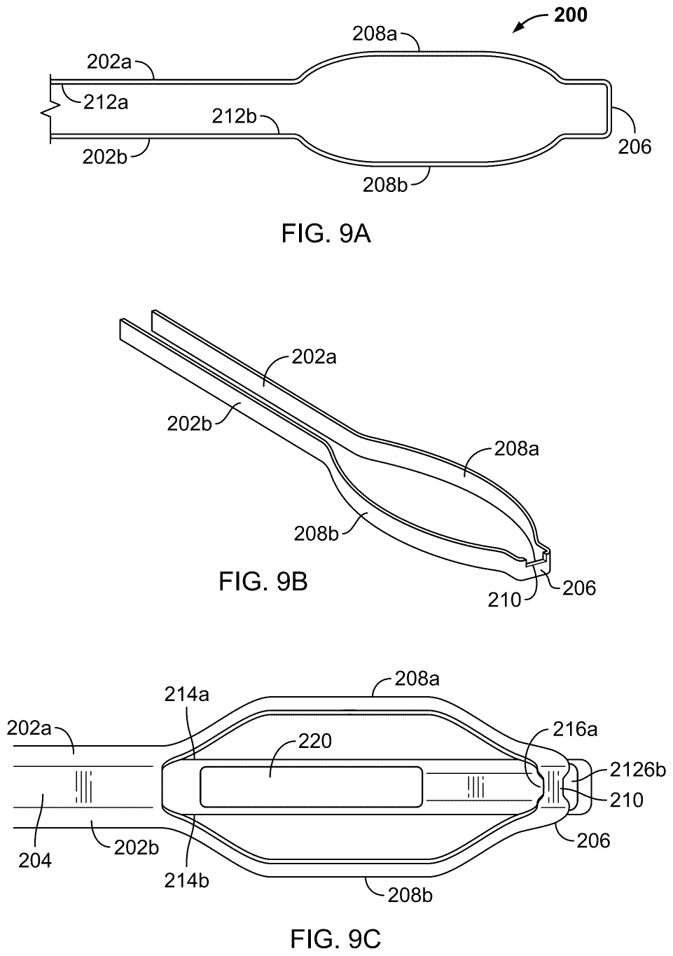

FIGS. 9A-9C illustrate an expandable member 200 that can be configured for attachment to the distal end of an elongate member. Expandable member 200 can include arms 202a and 202b situated in parallel (or substantially in parallel) and connected by a distal connecting member 206 at the distal end of member 200. Each arm can have a portion that expands outward past the longitudinal axis of arms 202a and 202b. Such expandable portions can be wings 208a and 208b. In some embodiments, wings 208a and 208b can connect to form a closed loop (not shown) in place of distal connecting member 206. Distal connecting member 206 can be grooved, channeled, or otherwise hollowed to create an opening 210 formed therein.

Wings 208a and 208b can be configured to be biased to an expanded position. In this embodiment, wings 208a and 208b are moved to a retracted position by moving distal connecting member 206 and/or arms 202a and 202b in a longitudinal direction away from wings 208a and 208b. In another embodiment, wings 208a and 208b are configured in a retracted position and are moved to an expanded position by moving distal connecting member 206 and/or arms 202a and 202b in a longitudinal direction toward wings 208a and 208b. In some embodiments, each wing can be expanded or retracted independently by moving the arm on the same side as the wing in a longitudinal direction away from or toward the wing as the configuration requires.

The proximal end of expandable member 200 can be attached to an elongate tissue access assembly 204 by connecting arms 202a and 202b to the shaft of the elongate assembly. In some embodiments, elongate assembly 204 is inserted through expandable member 200 such that the inwardly-facing side 212a of arm 202a and inwardly-facing side 212b of arm 202b are flush with a portion of the outwardly-facing sides 214a and 214b of elongate assembly 204 and secured via welding or other conventional means of attachment. The distal end of expandable member 200 can interact with a portion of the distal end of elongate assembly 204. For example, opening 210 can be configured to snap into, or be otherwise secured by, elongate assembly 204. As illustrated in FIG. 9C, elongate assembly 204 can include tabs 216a and 216b removably fastened over distal connecting member 206 thereby holding the distal end of expandable member 200 in place. In some embodiments, such as when elongate assembly 204 includes an aperture 220, wings 208a and 208b can be positioned in any plane relative to the aperture. Further, wings 208a and 208b may be in the same plane with respect to each other or may be positioned at an angle with respect to each other. In some embodiments, the angle between the plane of the wings is adjustable. In some embodiments, the elongate assembly is configured such that a treatment assembly, such as a needle assembly, is extendable through the aperture.

In some embodiments, expandable member 200 can be delivered using a sheath. The sheath functions to house expandable member 200 and collapse or compress wings 208a and 208b as the device is delivered to an interventional site of a patient. Once the target site is reached, extension of expandable member 200 through the distal opening of the sheath can be actuated by the handle assembly of the treatment device. As expandable member 200 exits the sheath, wings 208a and 208b expand or spring open, move away from the midline, to contact and manipulate target tissue. This delivery maintains wing alignment while preventing tissue trauma due to the wing edges. According to one embodiment, arms 202a and 202b are fixedly secured to elongate assembly 204 and distal connecting member 206 is free to move longitudinally with respect to wings 208a and 208b to enable expansion and retraction of wings 208a and 208b as they exit the sheath. According to another embodiment, arms 202a and 202b are free to move longitudinally with respect to wings 208a and 208b and distal connecting member 206 is fixedly secured to elongate assembly 204 to enable expansion and retraction of wings 208a and 208b as they exit the sheath.

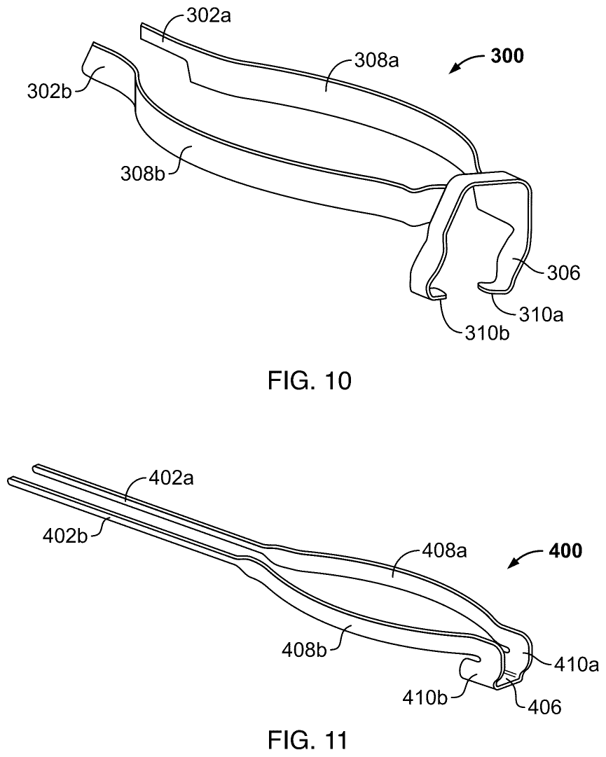

FIG. 10 shows another embodiment of an expandable member that can be attached to the distal end of a treatment device. Expandable member 300 can include arms 302a and 302b situated in parallel or substantially in parallel. Each arm can include a portion that is capable of expanding outward past the longitudinal axis of arms 302a and 302b. Wings 308a and 308b can be connected to a distal attachment piece 306 at the distal end of expandable member 300. In one embodiment, distal attachment piece 306 is horseshoe-shaped. Distal attachment piece 306 can be situated in a substantially perpendicular configuration with respect to wings 308a and 308b. Distal attachment piece 306 can include tabs 310a and 310b. Attachment piece 306 can be configured to receive and form a secure connection with a portion of the distal end of a treatment device.

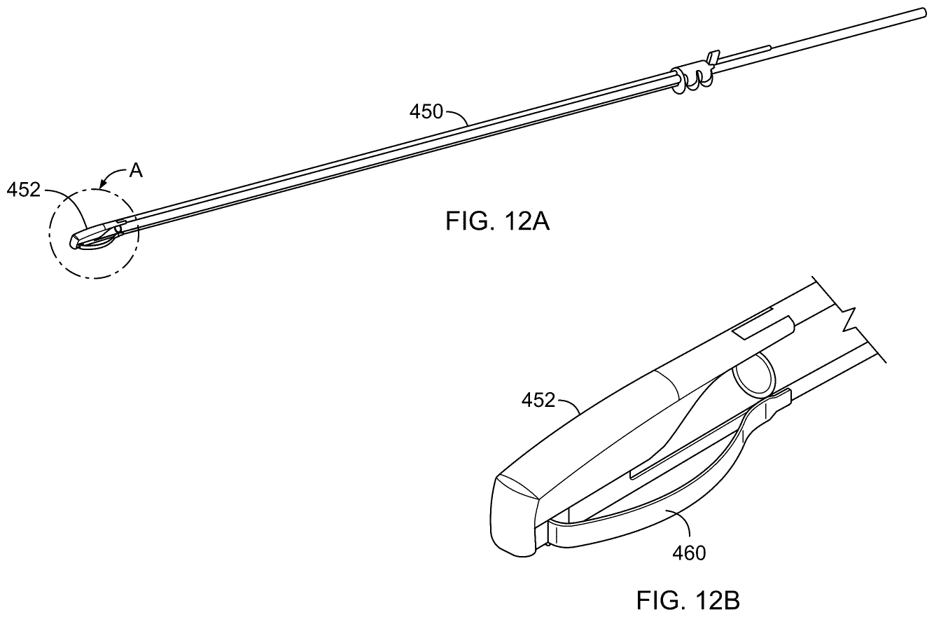

FIG. 11 shows another embodiment of an expandable member. Expandable member 400 can include arms 402a and 402b situated in parallel or substantially in parallel. Each arm can include a portion that is capable of expanding outward past the longitudinal axis of arms 402a and 402b. Wings 408a and 408b can terminate with curved ends 410a and 410b, respectively, that connect to create channel 406. Channel 406 can be configured to receive and secure a portion of the distal end of a treatment device.

FIGS. 10 and 11 illustrate configurations of the distal end of the expandable member that facilitate attachment of the expandable member to a treatment device. Other configurations that facilitate attachment of the expandable member to a treatment device are contemplated and the disclosure herein is not limited to the attachment configurations depicted in FIGS. 10 and 11.

Referring again to the embodiment of a treatment device depicted in FIGS. 8A-B, the device is configured such that the needle actuator 108 and the needle retracting lever 110 are in a ready position capable of providing treatment, such as delivery of a tissue anchor. Upon depression of the needle actuator 108, the needle assembly 106 is advanced from within the elongate tissue access assembly 104 (See FIG. 8B). The needle assembly can be configured so that it curves back toward the handle as it is ejected. In use in a prostate intervention, the needle assembly is advanced through and beyond a prostate gland. Spring deployment helps to ensure the needle passes swiftly through the tough outer capsule of the prostate without “tenting” the capsule or failing to pierce the capsule.

After depression of the needle actuator 108 and the unlocking of the needle retraction lever 110, the needle retraction lever 110 can be actuated. Such action results in a withdrawal of the needle assembly 106. In some embodiments, the needle assembly 106 is withdrawn further than its original position within the device pre-deployment. In a prostatic interventional procedure, this action can be used to deliver an implant or various activatable members, such as a tissue anchor, to facilitate modification of prostatic tissue.

The expandable member can be used to position elongate tissue access assembly 104 such that it engages the median lobe prior to and during deployment of an implant and/or modification of prostatic tissue by increasing the surface area of the distal end of assembly 104. The expandable member can also be used to displace and widen the urethral wall.

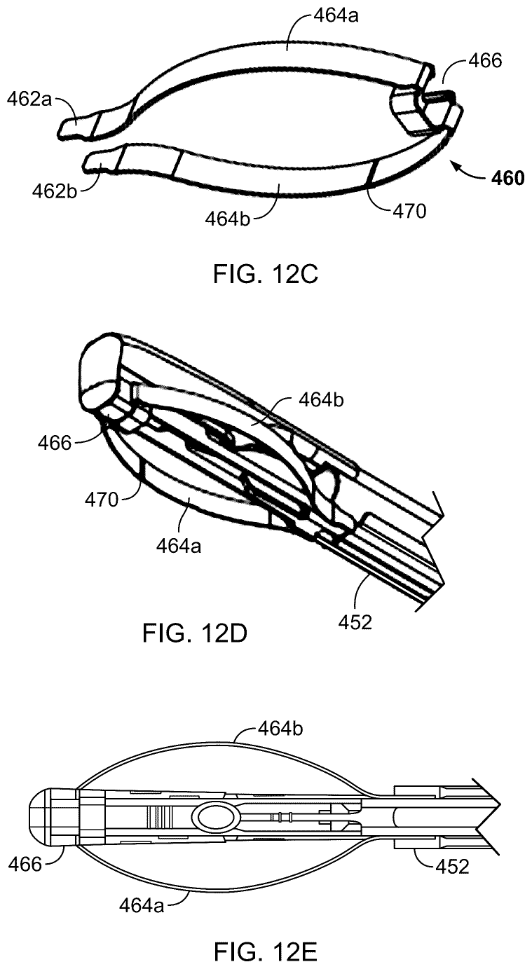

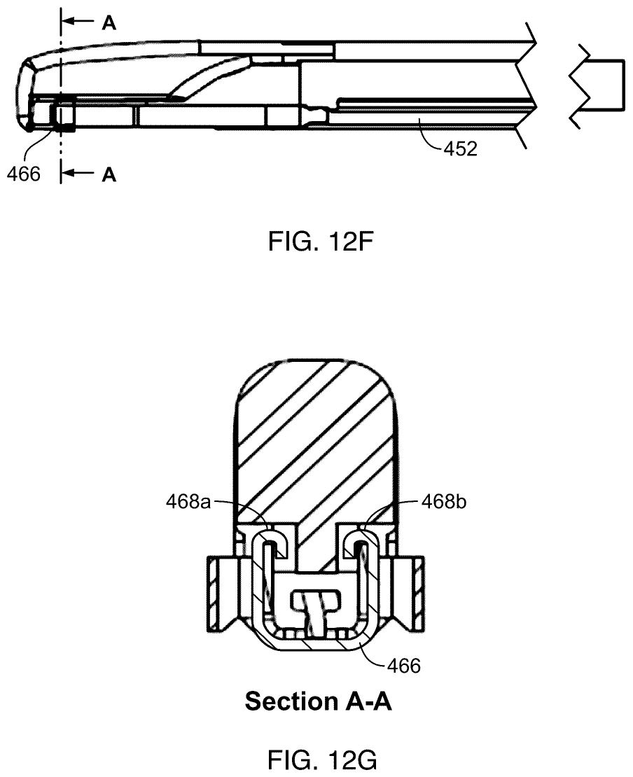

As shown in FIGS. 12A-G, an elongate assembly 450 can include a distal end 452 with an expandable member 460. Expandable member 460 can include arms 462a and 462b situated in parallel and secured to the shaft of distal end 452. Each arm can include an expandable portion, wings 464a and 464b, that expand outward past the longitudinal axis of arms 462a and 462b. The distal end of expandable member 400 can include channel 466 to receive and secure a portion of the distal end of elongate assembly 450. FIGS. 12F & 12G illustrate a cross-section of channel 466 with tabs 468a and 468b that fasten over a portion of the distal end 452 of elongate assembly 450. Insertion of the elongate assembly 450 into channel 466 allows the distal end of expandable member 460 to slide along the shaft of the assembly and facilitate wing position from a “closed” (compressed toward the midline) to an “open” (displaced away from the midline) configuration. Additionally, movement of expandable member 460 relative to the treatment device is constrained to the longitudinal axis of the treatment device. Furthermore, such an alignment presents no additional obstruction to cystoscope or endoscope view when such viewing devices are used during a procedure.

When elongate assembly 450 is inserted into a sheath (not shown), such as when the device is delivered to an interventional site of a patient, wings 464a and 464b reduce in profile by collapsing toward the midline of the longitudinal axis of the distal end the treatment device. Once the target site is reached, wings 464a and 464b can expand away from the midline of the treatment device when the elongate assembly 450 is extended through the distal opening of the sheath. Wings 464a and 464b are then available to engage and manipulate tissue.

In some embodiments, expandable member 460 is made from stainless steel having a 0.006-inch (0.015 cm) thickness. It can be advantageous for the cross-section of the arms and or wings of the expandable member to be asymmetrical. For example, the cross-section can be rectangular or elliptical such that one axis is substantially longer then its orthogonal axis. The purpose of this asymmetry is to provide flexibility in one direction and stiffness in the orthogonal direction. The stiffness facilitates capture and manipulation of tissue, while flexibility facilitates expansion and retraction of the expandable member. In some or the same embodiments, the arms and/or wings are configured with rounded edges to minimize tissue trauma during use. In some embodiments, the internal face of wings 464a and 464b can include visual line marker(s) 470 that indicate an entry position for a needle assembly (not shown) that exits from the side of elongate assembly 450.

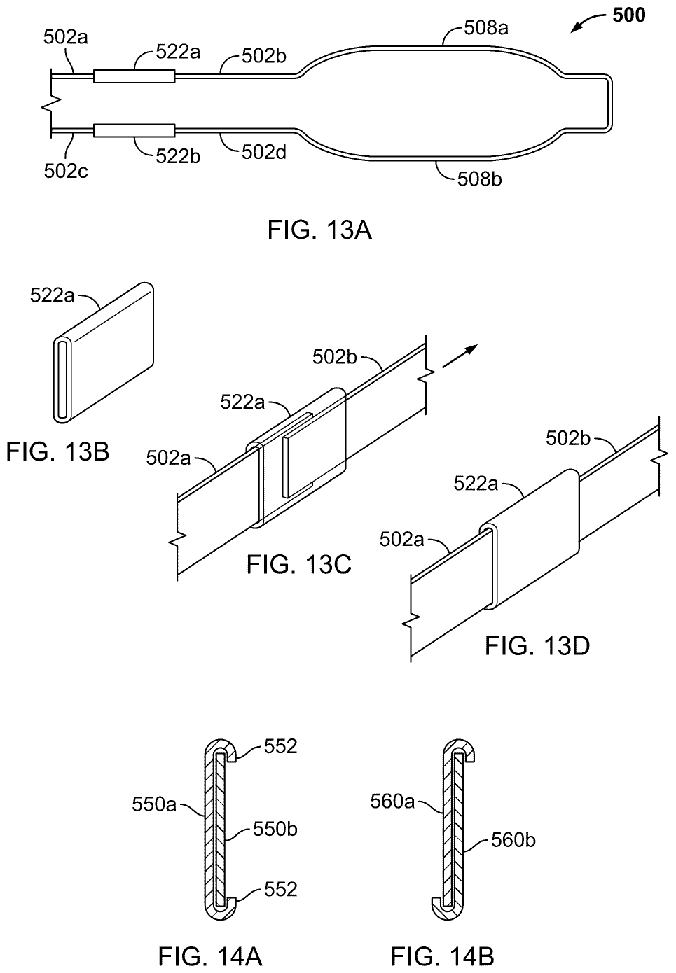

Other embodiments of structures that can be used to better engage and manipulate target tissue, including the median lobe of the prostate, are contemplated below. In some embodiments, wings 508a and 508b can be deployed from an expandable member 500 using a pair of telescopic arms. As shown in FIGS. 13A-13D, wing 508a is attached to or continuous with arm 502b. Arms 502a and 502b can be coupled within sleeve 522a such that arm 502b glides along stationary arm 502a (indicated by the dashed arrow in FIG. 13C) to transition from a contracted to an expanded state while simultaneously transitioning wing 508a outward from the longitudinal axis of member 500 from a compressed (“closed”) to an expanded (“open”) state. Expandable member 500 can be inserted into a sheath for placement at an interventional site. Once the device is positioned at the target site, the delivery device can be actuated to deploy arms 502b and 502d from sleeves 522a and 522b, respectively, to engage target tissue.

Alternative embodiments of telescopic arms are illustrated in FIG. 14A & 14B. In FIG. 14A, interlocking arms 550a and 550b are shown. Arm 550a can house arm 550b and include rounded ends 552 that cover the top and bottom edges of arm 550b. In FIG. 14B, each of interlocking arms 560a and 560b have a rounded end and a flat end. When interlocked, the rounded end of each arm covers the flat end of the opposite arm.

In the telescopic arm design of FIGS. 13A-13D and FIGS. 14A & 14B, the stationary arms can be secured to the shaft of the elongate assembly of the delivery device. Such a design provides guided, stabilized alignment and delivery of wings, or a similarly looped distal end, to an interventional site.

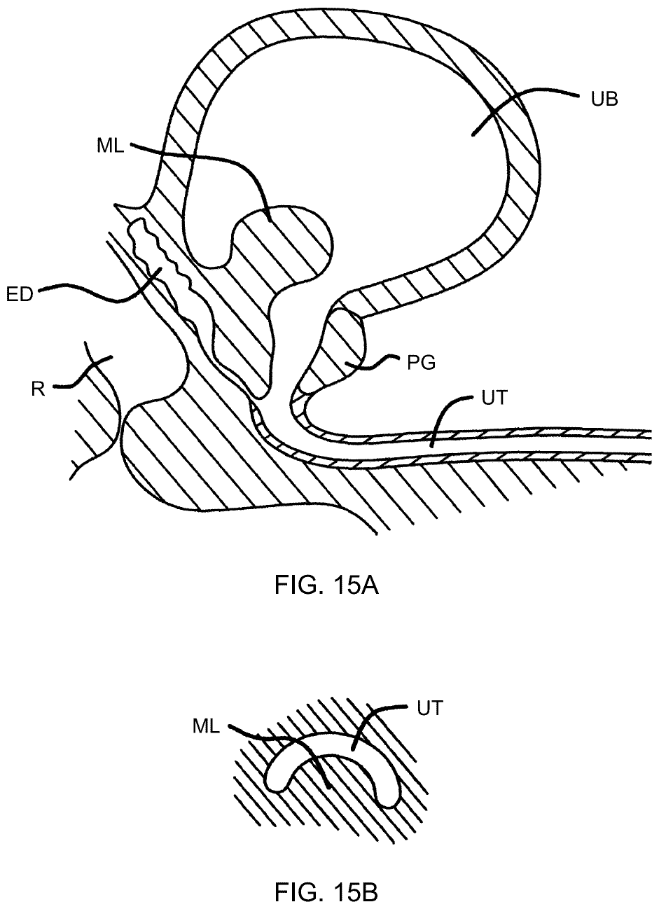

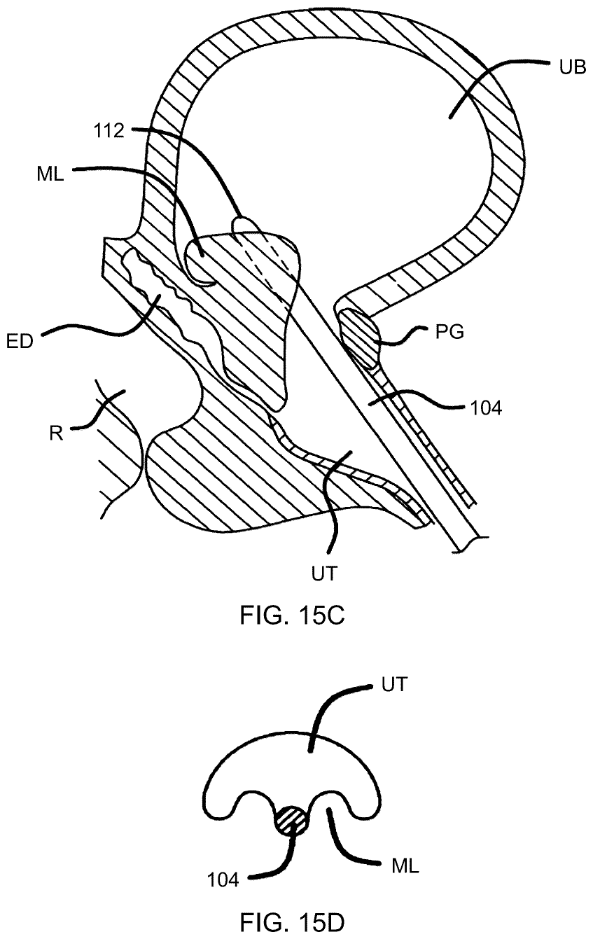

Turning now to FIGS. 15A-D, an approach to engaging and manipulating an enlarged median lobe ML is presented. Such an approach can be used as a complementary therapy with treatments for lateral lobes or can be employed solely to treat a median lobe ML. Because an enlarged median lobe ML can extend into the urinary bladder UB and may act as a ball valve interfering with normal function (See FIGS. 15A and 15B; FIG. 13B is a view through the prostatic urethra and into the urinary bladder), moving tissue away from a ball valve location (that is, away from the bladder neck) may be desired. By avoiding such invasive approaches (such as TURP), there is significantly less risk of disrupting the nerve tissue and/or the smooth muscle of the bladder neck. With less disruption to these tissues, ejaculating function and continence complications will likely be lower.

Accordingly, an approach involving inserting a device into the prostatic urethra UT transurethrally to compress and/or displace the median lobe ML is contemplated. Once the lobe is compressed or displaced, other procedures such as implanting tissue anchors or implants in a specific direction to maintain the compression/displacement of the median lobe.

As an initial step, sagittal views of a patient's bladder and prostate can be taken using transabdominal or transrectal ultrasonography. In this way, the patient's anatomy can be assessed. In this regard, an intravesical prostate measurement is taken to determine the vertical distance from a tip of the median lobe protrusion to the base of the bladder. As shown in FIGS. 15C-D, after assessing the anatomy, the elongate tissue access assembly 104 of an anchor delivery device (See FIGS. 8A and 8B) is advanced within the urethra UT and into apposition with the median lobe ML. FIG. 15D is a view through the urethra UT depicting the compression and displacement of the median lobe ML.

One specific series of actions is to position the tissue access assembly 104 so that its terminal end 112 is anterior to a prominent portion of the median lobe ML and then displace the surface in the posterior direction to move the median lobe ML away from a centerline of the urethra lumen UT. The median lobe consequently forms a tissue fold (Sec FIG. 15D) about the delivery instrument. In embodiments in which an expandable member is used to engage and manipulate the median lobe, the expandable member provides increased surface area as compared to tissue access assembly 104 in FIG. 15D, which facilitates temporary capture of the median lobe so that it can be displaced. In some embodiments, tissue of the median lobe can be captured in the space between the wings of the expandable member and the distal end of the elongate tissue treatment device. Thus, the wings of the expandable member can be configured to promote such capture, including by varying the amount of expansion that the wings undergo during deployment and retraction.

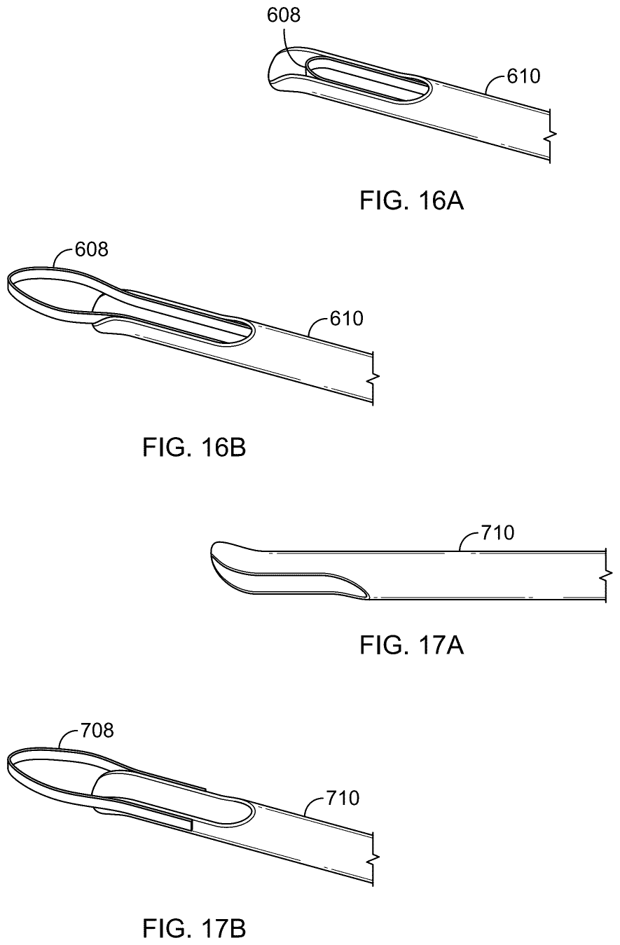

FIG. 16A & 16B illustrate another embodiment of a looped or expandable member. In this embodiment, a looped member 608 can be housed in the distal end of a sheath 610 such that the expandable wing portion of looped member 608 is compressed. When an elongate assembly is passed through sheath 610, it contacts the proximal end of looped member 608 (not shown) and pushes the wing portion out of sheath 610, allowing it to expand and manipulate target tissue.

In some embodiments, a looped member 708 can be attached to the distal end of a sheath 710, as shown in FIGS. 17A & 17B. In these embodiments, looped member 708 can expand and/or manipulate tissue at an interventional site prior to deployment of an elongate assembly. In some embodiments, looped member 708 can be made of a level ribbon, that is, having a linear cross-section. In other embodiments, looped member 708 can be made of a semi-circular ribbon having a c-shaped cross-section to prevent the edges of the device from contacting patient tissue during delivery.

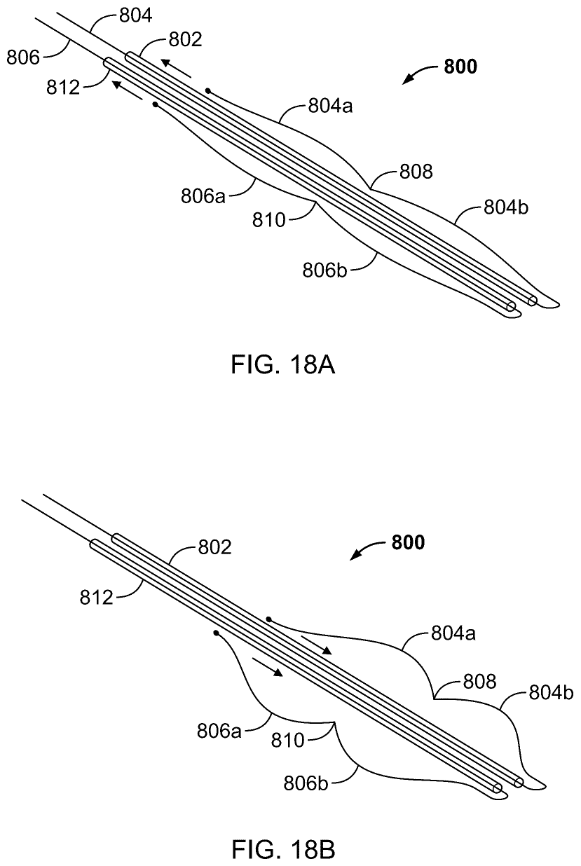

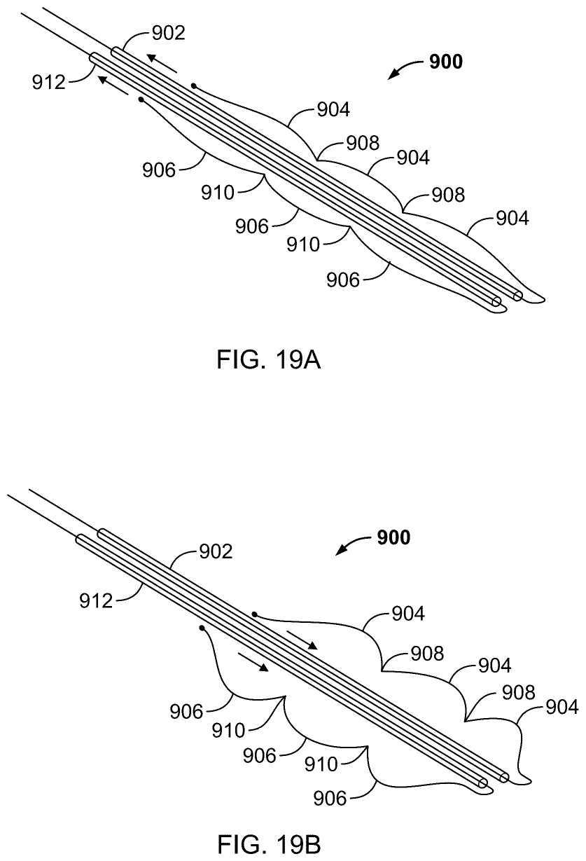

Alternative embodiments of an expandable member are shown in FIGS. 18A & 18B and 19A & 19B. Expandable member 800 can include a pair of tubes 802 and 812 attached to opposite sides of the shaft of an elongate assembly of a delivery device (not shown). Such tubes can be biased to the distal end of the elongate assembly. Tubes 802 and 812 can house wires 804 and 806, respectively, which are threaded through the lumen of the tube and then exit the distal end of each tube. The wire that exists the tube is positioned substantially parallel to the outer length of each tube and is bent, bowed, arced, or otherwise curved. As shown in FIG. 18A & 16B, wire 804 includes a pair of expandable portions 804a and 804b with a cinched waist 808 therebetween and wire 806 includes a pair of expandable portions 806a and 806b with a cinched waist 810 therebetween. In some embodiments, the cinched waist of each wire can be a compression spring coil that allows the expandable portions to collapse toward or expand from the midline of the elongate assembly. FIG. 18A illustrates expandable member 800 in a compressed or collapsed state, such as when the elongate assembly is housed within a sheath (not shown). FIG. 18B illustrates expandable member 800 in an expanded state, such as when the elongate assembly exits the distal end of a sheath (not shown) freeing the springs to lengthen and, in turn, move the expandable portion of each wire away from the midline of the treatment device. FIGS. 19A and 19B illustrate another embodiment of expandable wire structures that function similar to expandable member 800, but expandable member 900 includes three expandable portions.

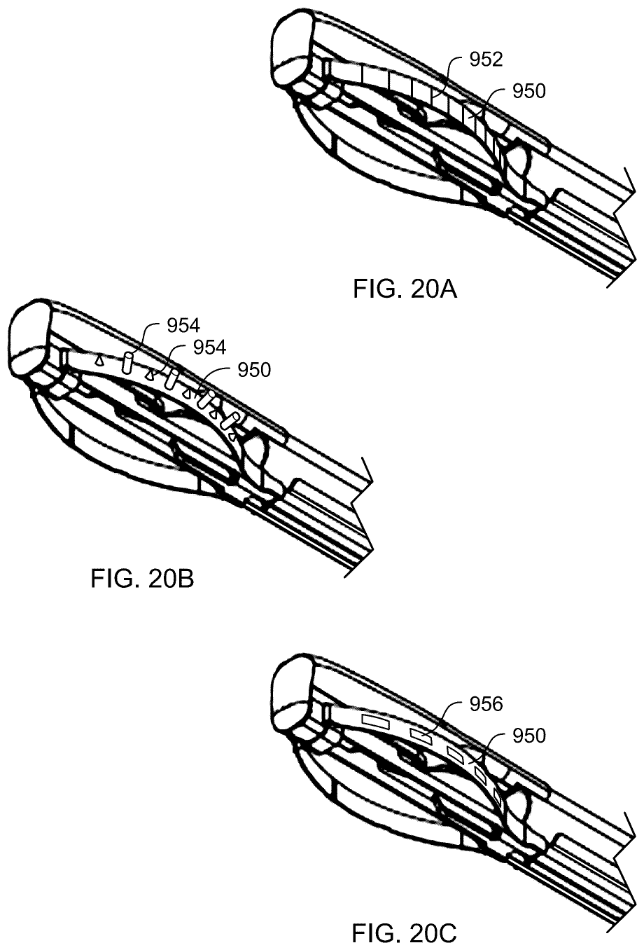

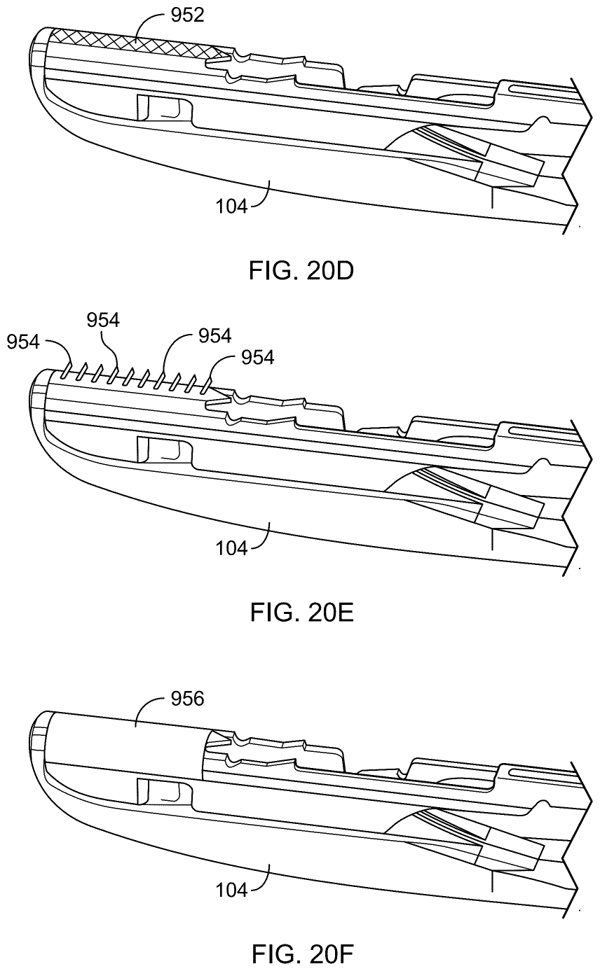

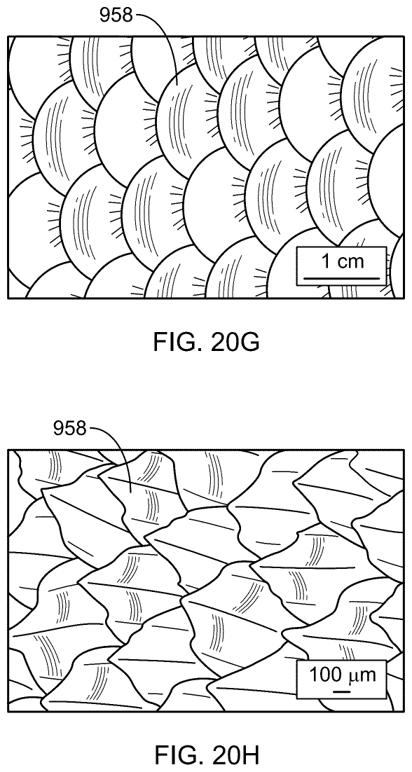

Various approaches are contemplated for best engaging median lobe or other tissue. Additional structural features can be incorporated into the distal end of the expandable member of treatment device 100 for the purpose of increasing frictional forces between the target tissue and a distal portion of the treatment device 100, or for increasing the surface area of the treatment device. Knurled or roughened surfaces 952 can form surface components of portions or an entirety of the winged or expandable portions 950 (See FIG. 20A) of the treatment device 100. In some embodiments, as shown in FIG. 20B one or more of spikes, fangs, hooks, barbs or other protuberances 954, or a combination thereof can be configured to extend at various angles and lengths from expandable portion 950. Such protuberances can be sharp or blunted. Such structures can be one or more of retractable, flexible or fixed. Thus, in one or more approaches, these structures can be associated with a pop-up feature attached to a puller (not shown). In one aspect, control of the of the pop-up feature can be achieved with side actuators to deploy and retract extendable tissue engaging and manipulation features when slid down the sheath and a spacer to change a relationship between the sheath and shaft for controlling deployment of the side actuators. In one particular approach, the spikes, fangs, hooks, barbs, or protuberances can be angled proximally so that enhanced tissue engagement is provided when withdrawing the delivery device and is released when advancing the delivery device.

Atraumatic tape can be placed over the spikes, fangs, hooks, barbs, or protuberances 954 prior to use of the treatment device 100. The tape can be removed prior to performing a median or middle lobe procedure. It is thus contemplated that the treatment system can be provided in two configurations. A first configuration can be for normal use such that it includes atraumatic tape that covers sharp or other tissue engaging features and prevents them from interacting with the tissue. A second configuration can be for median lobe engagement and manipulation usage, where the system is assembled and shipped without atraumatic tape covering sharp or other tissue engaging features.

In some embodiments, tissue adhesive material 956 can be added to the exterior surface at various locations along expandable portion 950 (See FIG. 20C). In certain contemplated approaches, such tissue adhesive material 956 can range from adhesive tape to material that is swellable in fluid such that it changes its adhesion property when desired. Another contemplated approach to adhesion upon pressure involves scale-like projections 958 (FIGS. 20F & 20G) incorporated onto expandable portion 950. High friction is created when pulling the target tissue in a direction against the scales, but no or little or less friction after deployment or when employing the treatment device 100 with the direction of scales 958. Here also, such structure can be one or more of retractable, flexible or fixed.

Structures that provide enhanced frictional or other engagement forces also can be based upon an adhesive that responds to pressure. For example, the expandable distal portion of treatment device 100 can additionally or alternatively include micro-hooks similar to Velcro technology, the same requiring tissue to be pressed against it to create a secure engagement.

As stated, such tissue engaging structures can be placed in various locations along the expandable portion of treatment device 100. Textures or protuberances can be configured to engage tissue such that tissue will roll with the distal end of the device as it is rotated, for example, into a deployment position. In some embodiments, the structures can be located on side areas of the treatment device where there is more space. That is, these structures can be located away from the exit points for the therapeutic elements that may extend from the treatment device. It is specifically contemplated that structures can be located along a “tissue contacting fence.” That is, protuberances can be hidden from tissue contact in a “tissue contacting fence” and configured to extend beyond this protective fence via a user operated actuator.

Referring now to FIG. 20D-F, elongate tissue access assembly 104, and in particular the distal end of elongate tissue access assembly 104, can include knurled or roughened surfaces 952, spikes, fangs, hooks, barbs, or protuberances 954 (and, optionally, atraumatic tape), and/or tissue adhesive material 956. In certain embodiments, one or both of the elongate tissue access assembly and the expandable member can contain one or more of these features. That is, the embodiments described above, illustrated in FIGS. 20A-H, can be incorporated into treatment device 100 to supplement, or enhance, the tissue engagement and manipulation capacity of the expandable or winged portion of the device, of the distal end of the elongate tissue access assembly, or both.

Vacuum forces can also be employed to facilitate engaging and manipulating tissue. In this regard, a suction or vacuum source (not shown) can be incorporated into the expandable portion or attached thereto, and a channel provided to communicate with the distal end of the treatment device 100. In this way, the vacuum forces can be initiated and controlled when the treatment device 100 is positioned to engage and manipulate target tissuc.

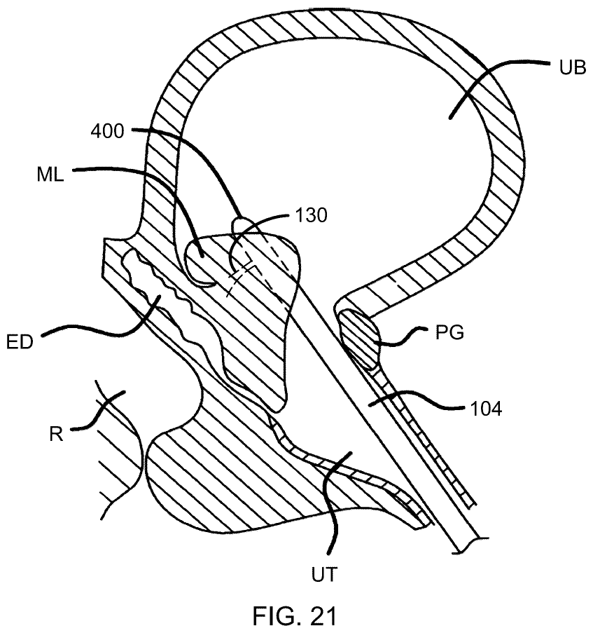

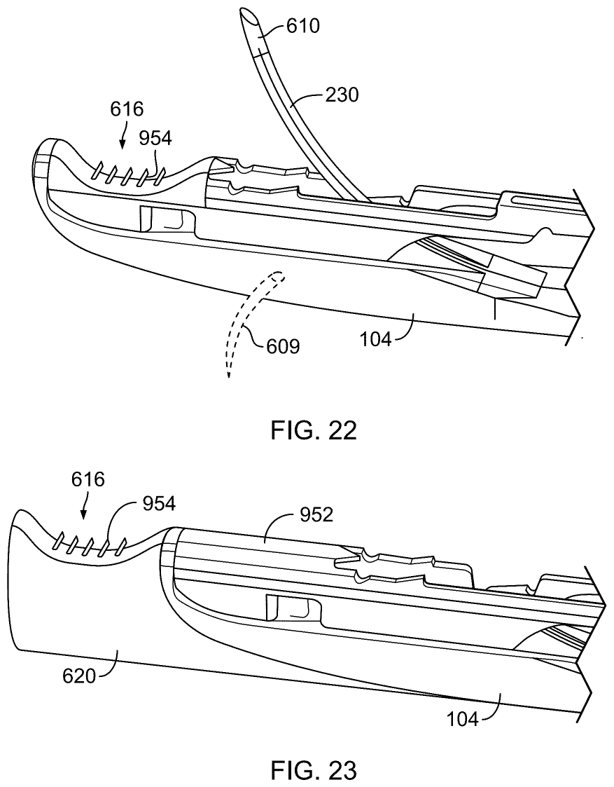

As shown in FIG. 21, in another approach, partial controlled deployment of the needle 230 can be utilized to engage and manipulate target tissue. The needle 230 can be retracted for later full deployment associated with, for example, implantation of an anchor device. Additionally or alternatively, one or more supplemental, side-projecting needles 609 (shown in dashed lines in FIG. 22) can be provided for engaging and manipulating purposes. Various approaches to reinforcing the distal portion of the needle 230 also contemplated so that a more robust structure is presented for tissue manipulation. For example, a supplemental needle tip 610 can be attached to a terminal and portion of the needle 230. In one contemplated approach, the needle tip 610 can be attached to structure that is configured to be actuated from the delivery device handle. The needle 610 can define a solid member to thereby provide sufficient mechanical strength for manipulating tissue, and be connected to any elongated member that extends within the delivery device which extends along and outside of the delivery device but within an introducer sheath or through fluid holes therein. Manual or automated approaches to control the use or removal of the needle tip 610 are both envisioned.

With reference to FIG. 22, the distal end portion 104 of the delivery device 100 can also, additionally or alternatively, include a divot or recess having a radius of curvature that matches, or generally receives, the contours of the median lobe or other target tissue. The interior of the recess can be configured with any of the described structures for engaging and manipulating tissue. For example, FIG. 22 illustrates recess 616 as including a plurality of spikes 954. Referring now to FIG. 23, a second or supplementary sheath 620 can be provided and configured about the distal portion 104 of delivery device 100. Sheath 620 itself can include one or more of the tissue engaging and manipulating features described herein. Such features can be reserved for one or more sides or portions of the sheath 620 or can be positioned completely around sheath 620. Additionally, sheath 620 itself can include recess 616 configured to match tissue anatomy. Thus, recess 616 can be included in distal end 104 of delivery device 100 by fabricating distal end 104 with a recess 616, or by using sheath 620 that have been fabricated with a recess 616.

The target tissue or median lobe specifically can be pre-treated to facilitate engagement with the treatment device 100. In this regard, it is contemplated that the target tissue can be subjected to electro-cauterization, botox or other modality to alter its mechanical profile. The target tissue can alternatively or additionally be pre-treated by making incisions therein. Finally, the target tissue can be lassoed to support the tissue or to accomplish the desired manipulations.

It is to be recognized that various materials are within the scope of the present disclosure for manufacturing the disclosed devices. Moreover, one or more components disclosed herein can be completely or partially biodegradable or biofragmentable.

Further, as stated, the devices and methods disclosed herein can be used to treat a variety of pathologies in a variety of lumens or organs comprising a cavity or a wall. Examples of such lumens or organs include, but are not limited to urethra, bowel, stomach, esophagus, trachea, bronchii, bronchial passageways, veins (e.g. for treating varicose veins or valvular insufficiency), arteries, lymphatic vessels, ureters, bladder, cardiac atria or ventricles, uterus, fallopian tubes, etc.

Finally, it is to be appreciated that the disclosure has been described hereabove with reference to certain examples or embodiments of the disclosure but that various additions, deletions, alterations and modifications may be made to those examples and embodiments without departing from the intended spirit and scope of the disclosure. For example, any element or attribute of one embodiment or example may be incorporated into or used with another embodiment or example, unless to do so would render the embodiment or example unpatentable or unsuitable for its intended use. Also, for example, where the steps of a method are described or listed in a particular order, the order of such steps may be changed unless to do so would render the method unpatentable or unsuitable for its intended use. All reasonable additions, deletions, modifications and alterations are to be considered equivalents of the described examples and embodiments and are to be included within the scope of the following claims.

Briefly and in general terms, the present disclosure is directed towards an apparatus and method for engaging and manipulating internal body structures. Such engagement and manipulation can form the primary or alternatively, form a supplementary or integral part of a multi-step interventional procedure. In one aspect, the apparatus includes elongate member configured to engage tissue in order to manipulate or reposition that tissue. In some embodiments, the tissue is a prostate. In some embodiments, the tissue is the median lobe of a prostate.

In various approaches, the apparatus can include a portion that is equipped with structure that increases frictional forces between the apparatus and tissue to be manipulated. The apparatus can additionally or alternatively include an extendable needle that can be partially deployed to engage or manipulate target tissue. A retractable sheath can also additionally or alternatively be provided to facilitate engaging and manipulating tissue. Further, the surface area of a distal end portion of the apparatus can include structure(s) intended to increase surface area and thus present structure(s) specifically configured to effectively engage or manipulate tissue. Moreover, the apparatus can be configured and employed to pre-treat target tissue by subjecting the tissue to energy or substances that alter the mechanical profile or by creating incisions therein.

Thus, the delivery apparatus of the present disclosure can additionally include various subassemblies which are mobilized via an actuator or other manually accessible structure. The operation of the subassemblies is coordinated and synchronized to ensure accurate and precise navigation and placement of the tissue engaging or manipulation structure.

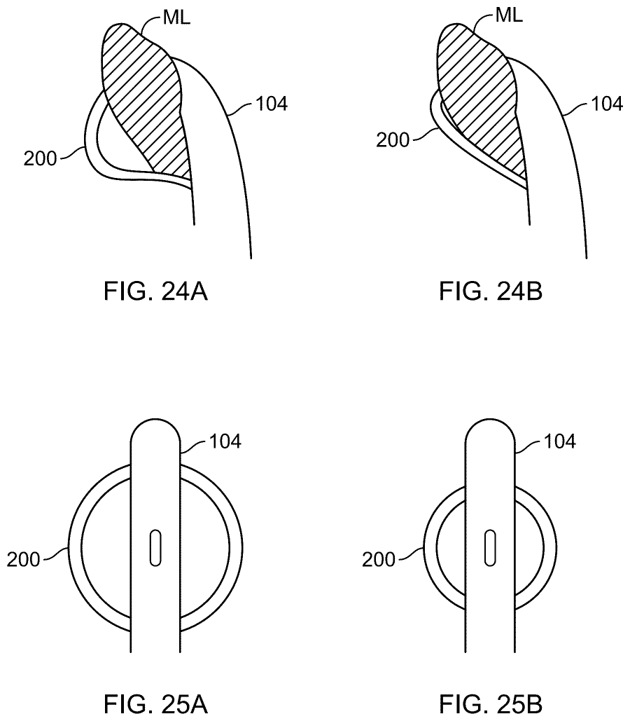

Referring now to FIG. 24A-B, in some embodiments expandable member 200 is adjustable. FIGS. 24A and 24B illustrate a distal end of elongate tissue access assembly 104 and expandable member 200 present on one side of elongate tissue access assembly 104. That is, in this particular embodiment there is only one set of arms or wings. FIG. 24 A illustrates expandable member 200 in an expanded state, while FIG. 24B illustrates expandable member 200 in a contracted state. Median lobe ML can be captured within the space between expandable member 200 and elongate tissue access assembly 104. Then, expandable member 200 can be contracted or cinched down such that median lobe ML is secured within the space between expandable member and elongate tissue access assembly 104. In this position, median lobe ML can be manipulated and/or displaced.

Referring now to FIG. 25A-B, expandable member 200 can be adjusted from a larger size (FIG. 25A) to a smaller size (FIG. 25B) in embodiments in which expandable member 200 includes two or more sets of arms and wings. In these embodiments, expandable member 200 can be used to capture tissue in the space between expandable member 200 and elongate tissue access assembly 104, or expandable member 200 can be used to manipulate and/or displace tissue according to other embodiments disclosed herein. The individual arms of expandable member 200 can be adjusted individually or together.

In the embodiments in which expandable member 200 can be adjusted, it is contemplated that the adjustments can occur prior to putting the device in the patient (or prior to putting the device within the sheath in the embodiments in which a sheath is used) or the adjustments can occur at or near the site of tissue manipulation within the patient. A physician can set the size of expandable member 200 based on the patient's anatomy, for example. The physician may be able to determine the desired size for expandable member 200 based on measurement and/or observation of the patient's anatomy prior to and/or during a procedure.

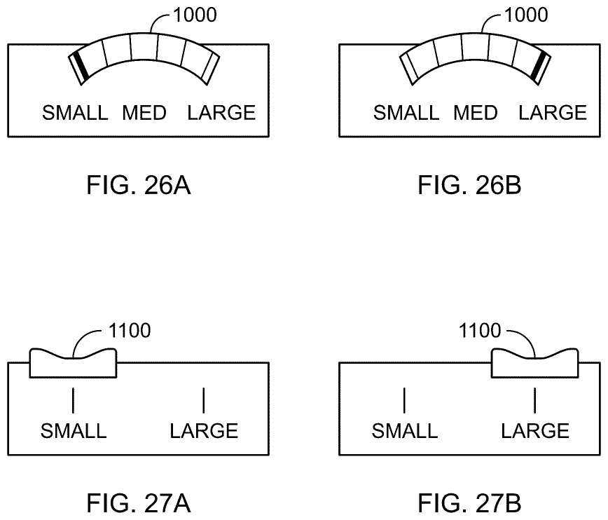

Referring now to FIG. 26A-B and 27A-B, the handle of device 100 can include wheel 1000 or slider 1100, each of which are coupled to expandable member 200 and configured to expand or contract expandable member 200. In certain embodiments, wheel 1000 and slider 1100 provide continuous adjustability from a “Small” expansion size to a “Medium” expansion size to a “Large” expansion size, where the extent of expansion is indicated with a tactile indicator. Optionally, a lock is provided on wheel 1000 or slider 1100 such that the size of the expandable member can be fixed. In certain other embodiments, wheel 1000 and slider 1100 provide discrete adjustability from a “Small” expansion size to a “Medium” expansion size to a “Large” expansion size. That is, there are two or more pre-set sizes for expandable member 200 and wheel 1000 or slider 1100 allow for the physician to elect between or among those sizes only.

Various alternative methods of use are contemplated. The disclosed apparatus can be used to facilitate improving flow of a body fluid through a body lumen, modify the size or shape of a body lumen or cavity, treat prostate enlargement, treat urinary incontinence, support or maintain positioning of a tissue, close a tissue wound, organ or graft, perform a cosmetic lifting or repositioning procedure, form anastomotic connections, and/or treat various other disorders where a natural or pathologic tissue or organ is pressing on or interfering with an adjacent anatomical structure. Also, the disclosure has a myriad of other potential surgical, therapeutic, cosmetic or reconstructive applications, such as where a tissue, organ, graft or other material requires approximately, retracting, lifting, repositioning, compression or support.

One aspect of the invention is a system for engaging and manipulating a median lobe of a prostate gland that includes a sheath and a tissue engaging or manipulation device housed within the sheath, the tissue engaging or manipulation device being sized and shaped to be inserted within a patient's urethra and to extend within prostate tissue, the tissue engaging or manipulation device including a moveable engagement structure that can transition from a compressed state to an expanded state to enhance contact with the median lobe, wherein the engagement structure is biased to a distal end of the tissue engaging or manipulation device.

In another aspect of the invention, the engagement structure comprises a first arm and a second arm anchored to opposite sides of a shaft of the tissue engaging or manipulation device.

In another aspect of the invention, the first arm comprises a first expandable portion and the second arm comprises a second expandable portion, wherein the first expandable portion and the second expandable portion compress toward the shaft when the tissue engaging or manipulation device is housed in the sheath.

In another aspect of the invention, the first expandable portion and the second expandable portion expand away from the shaft when the tissue engaging or manipulation device exits the sheath.

In another aspect of the invention, the engagement structure further comprises a channel to receive and secure a portion of the distal end of the tissue engaging or manipulation device.

In another aspect of the invention, when the distal end of the tissue engaging or manipulation device is secured in the channel, movement of the engagement structure is constrained to a longitudinal axis of the tissue engaging or manipulation device.

In another aspect of the invention, movement along the longitudinal axis of the tissue engaging or manipulation device confers the transition of the engagement structure from the compressed state to the expanded state.

In another aspect of the invention, the engagement structure is made from a ribbon with a plurality of round edges to reduce trauma to tissue, wherein the ribbon is asymmetrical in cross-section.

In another aspect of the invention, the engagement structure is made from a ribbon with a c-shaped cross-section that reduces trauma to tissue.

In another aspect of the invention, the shaft of the tissue engaging or manipulation device further comprises a side aperture and a needle assembly that exits from the side aperture, wherein the side aperture is aligned between the first expandable portion and the second expandable portion of the engagement structure.

In another aspect of the invention, the first expandable portion includes an internal face with a first visual line marker and the second expandable portion includes an internal face and a second visual line marker.

In another aspect of the invention, the first visual line marker and the second visual line marker of the engagement structure indicate the tissue entry position for the needle assembly after exiting the side aperture.

In another aspect of the invention, the first arm and the second arm of the engagement structure are configured to be telescopic.

In another aspect of the invention, the engagement structure is a loop affixed to a terminal portion of a shaft of the tissue engaging or manipulation device.

In another aspect of the invention, the loop is flexible and configured to compress toward the shaft when the tissue engaging or manipulation device is housed in the sheath and expand when the tissue engaging or manipulation device exits the sheath.

In another aspect of the invention, the engagement structure is adjustable prior to or during a procedure, and the adjustment can be continuous or discrete via a control device on the handle of the system.

In another aspect of the invention, the engagement structure is configured to enhance frictional contact with the median lobe.

In another aspect of the invention, the engagement structure includes one or more of spikes, fangs, hooks, barbs or other protuberances arranged at various angles and having various lengths.

In another aspect of the invention, the engagement structure is defined by a knurled surface.

In another aspect of the invention, the engagement structure is defined by an adhesive surface, which optionally may be swellable.

In another aspect of the invention, the engagement structure is defined by scales.

In another aspect of the invention the system includes a first projectable needle and a second projectable needle.

In another aspect of the invention, a reinforcing structure is affixed to a terminal and of one or more of the first and second project of needles.

In another aspect of the invention, the engagement structure includes and atraumatic tape that is configured to cover the engagement structure.

In another aspect of the invention, the engagement structure is defined by a divot formed on a portion of the distal end of the system, the divot sized and shaped to substantially fit a contour both target tissue.

In another aspect of the invention, tissue is pre-treated prior to being manipulated by an engagement structure, and the pre-treatment includes one or more of electro-cauterizing, botox, or incisions.

Thus, it will be apparent from the foregoing that, while particular forms of the disclosure have been illustrated and described, various modifications can be made without parting from the spirit and scope of the disclosure.

We claim:

1. A treatment device for engaging and manipulating a lobe of a prostate gland, comprising:
an elongate tissue access assembly coupled to a handle assembly, wherein the elongate tissue access assembly is configured to be inserted within an introducer sheath; and
an expandable tissue engagement structure coupled to a distal end portion of the elongate tissue access assembly, wherein the expandable tissue engagement structure comprises connected curved portions that form a closed loop structure configured to transition from a contracted state to an expanded state upon exiting a distal end of the introducer sheath.

2. The treatment device of claim 1, wherein the curved portions are biased away from a midline defined by a longitudinal axis of the elongate tissue access assembly such that the curved portions are compressed toward the midline when positioned within the introducer sheath and expand away from the midline when positioned outside the introducer sheath.

3. The treatment device of claim 1, wherein a proximal portion of the closed loop structure is coupled to the elongate tissue access assembly.

4. The treatment device of claim 1, wherein at least a portion of the expandable tissue engagement structure is configured to be visualized using a cystoscope.

5. The treatment device of claim 1, wherein movement of the closed loop structure is constrained to be along a longitudinal axis of the elongate tissue access assembly.

6. The treatment device of claim 1, wherein the closed loop structure comprises arms, and the curved portions comprise wings.

7. The treatment device of claim 6, wherein the curved portions, arms, or wings are connected by a distal connecting member.

8. The treatment device of claim 6, wherein the curved portions, arms, or wings have connected distal ends.

9. The treatment device of claim 1, wherein the closed loop structure is configured to engage and manipulate a prostate gland in the expanded state.

10. The treatment device of claim 1, wherein the elongate tissue access assembly is configured such that upon insertion within a urethra, at least a distal portion of the closed loop structure is positioned with a bladder.

11. A system for engaging and manipulating a lobe of a prostate gland, comprising:
an introducer sheath; and
an elongate tissue access assembly configured for insertion through the introducer sheath and comprising an expandable tissue engagement structure, wherein the expandable tissue engagement structure comprises connected curved portions that form a closed loop structure configured to transition from a contracted state to an expanded state upon exiting a distal end of the introducer sheath.

12. The system of claim 11, further comprising a cystoscope.

13. The system of claim 11, further comprising a handle assembly coupled to a proximal end of the elongate tissue access assembly.

14. The system of claim 11, wherein the curved portions are biased away from a midline defined by a longitudinal axis of the elongate tissue access assembly such that the curved portions are compressed toward the midline when positioned within the introducer sheath and expand away from the midline when positioned outside the introducer sheath.

15. The system of claim 11, wherein a proximal portion of the closed loop structure is coupled to the elongate tissue access assembly.

16. The system of claim 11, wherein movement of the closed loop structure is constrained to be along a longitudinal axis of the elongate tissue access assembly.

17. The system of claim 11, wherein the closed loop structure comprises arms, and the curved portions comprise wings.

18. The system of claim 17, wherein the curved portions, arms, or wings are connected by a distal connecting member.

19. The system of claim 17, wherein the curved portions, arms, or wings have connected distal ends.

20. The system of claim 11, wherein the elongate tissue access assembly is configured such that upon insertion within a urethra, at least a distal portion of the closed loop structure is positioned with a bladder.
